(19) United States
(12) Patent Application Publication
(10) Pub. No.: US 2021/0322742 A1
(43) Pub. Date: Oct. 21, 2021

(54) FLEXIBLE APPLICATOR HANDLE REGION

(71) Applicant: 3M INNOVATIVE PROPERTIES COMPANY, SAINT PAUL, MN (US)

(72) Inventors: ALAN R. DOMBROWSKI, WOODBURY, MN (US); ROBERT A. ASMUS, HUDSON, WI (US)

(73) Assignee: 3M INNOVATIVE PROPERTIES COMPANY, SAINT PAUL, MN (US)

(21) Appl. No.: 17/267,624

(22) PCT Filed: Aug. 9, 2019
(86) PCT No.: PCT/IB2019/056799

§ 371 Date: Feb. 10, 2021

Related U.S. Application Data
(60) Provisional application No. 62/720,694, filed on Aug. 21, 2018.

(51) Int. Cl. A61F 13/40

(52) U.S. Cl. CPC A61M 35/006; A61M 2210/04

(57) ABSTRACT

An applicator (100) including a hollow body (110), the hollow body including a distal portion (112), a proximal portion (114), an exterior surface (118), an interior surface (116), and an activation region (120), where the hollow body includes at least two longitudinal grooves (122) on at least one of the exterior surface and the interior surface in the activation region of the applicator, and where the hollow body has a first radial groove (124) in the activation region that is on at least one of the exterior surface or the interior surface. The applicator includes a frangible ampoule (160) inside the hollow body, the frangible ampoule configured to contain a solution. The applicator may include a head (140) having an absorbent component (150) capable of absorbing the contents of the frangible ampoule when the ampoule is fractured and can aid in application of the ampoule contents to the desired surface.

### TECHNICAL FIELD

The present disclosure relates to an applicator for applying a liquid to a surface. In particular, the present disclosure relates to an applicator including a handle having a flexible region.

### BACKGROUND

Barrier products are used to protect the skin of patients who suffer from a variety of conditions including, for example, urinary and fecal incontinence, skin occlusion, ileostomy and colostomy. The presence of high moisture and corrosive enzymes from intestinal fluids particularly can lead to devastating breakdown of the skin, which can lead to fungal infections and denuding and erosion of the skin. Some of the products which can be used to protect the skin from these challenges include cyanoacrylate monomer solutions, solutions that require special storage conditions due to their tendencies to degrade upon exposure to ambient conditions.

One method of maintaining the functionality of solutions including cyanoacrylate monomers is to seal such solutions in a container, such as a glass ampoule that can be crushed to release the solution shortly before use. Applicators for dispensing small aliquots of solution, e.g., 0.5 mL to 2 mL, which are particularly desirable when the area to be covered is less than 30 square inches (200 cm²) typically employ glass ampoules placed into plastic tubes with foam attached to them and do not have levers for crushing the ampoule. To provide for a safe device, a suitably rigid and/or thick plastic wall material must be used to form the plastic tube, but since the tube is generally pinched between the fingers to activate, these devices may be difficult for users to activate, especially with a single hand.

### SUMMARY

The disclosed applicator includes a handle having an activation region that is sufficiently flexible so that an ampoule inside the applicator body may be readily fractured when the applicator is held in a single hand, while at the same time providing adequate protection to the user from injury by preventing ampoule shards from piercing the applicator body.

In one aspect, the applicator comprises a hollow body. The hollow body includes a distal portion, a proximal portion, an outer surface, an inner surface, and an activation region. The hollow body includes two longitudinal grooves on at least one of the outer surface and the inner surface in the activation region. The hollow body has a first radial groove in the activation region that is on at least one of the outer surface and the inner surface.

Features and advantages of the present disclosure will be further understood upon consideration of the detailed description as well as the appended claims.

### BRIEF DESCRIPTION OF THE DRAWINGS

FIG. 1 shows a perspective side view of an applicator including an embodiment of an activation region of the present disclosure.

FIG. 2 shows a longitudinal cross-section of the applicator of FIG. 1.

FIG. 3 shows a radial cross-section through an applicator activation region having two longitudinal grooves in the outer surface where the grooves are 180° apart.

FIG. 4 shows a radial cross-section through an applicator activation region having two longitudinal grooves in the inner surface where the grooves are 180° apart.

FIG. 5 shows a radial cross-section through an applicator activation region having two sets of opposing longitudinal grooves in the outer and inner surfaces where the grooves are 180° apart.

FIG. 6 shows a radial cross-section through an applicator activation region having a radial groove in the outer surface of the applicator.

Repeated use of reference characters in the specification and drawings is intended to represent the same or analogous features or elements of the disclosure. It should be understood that numerous other modifications and embodiments can be devised by those skilled in the art, which fall within the scope and spirit of the principles of the disclosure. The figures may not be drawn to scale.

### DETAILED DESCRIPTION

### Definitions

The term “a”, “an”, and “the” are used interchangeably with “at least one” to mean one or more of the elements being described.

The term “and/or” means either or both. For example, “A and/or B” means only A, only B, or both A and B.

The terms “including,” “comprising,” and “having,” and variations thereof, are meant to encompass the items listed thereafter, and equivalents thereof, as well as additional items.

The terms “polymer” and “polymeric material” refer to both materials prepared from one monomer such as a homopolymer or to materials prepared from two or more monomers such as a copolymer, terpolymer, or the like. Likewise, the term “polymerize” refers to the process of making a polymeric material that can be a homopolymer, copolymer, terpolymer, or the like. The terms “copolymer” and “copolymeric material” refer to a polymeric material prepared from at least two monomers.

The terms “longitudinal” and “axial” are used to refer to a direction or axis that is generally parallel to a central longitudinal axis of an applicator and generally parallel to the overall direction of substance flow.

The terms “lateral” and “transverse” are used to refer to a direction or axis that is perpendicular to the central longitudinal axis the longitudinal direction.

The terms “vertical” and “normal” are used to refer to a direction or axis that is normal to both the longitudinal and lateral directions (or axes).

The term “proximal” and “distal” are used to represent longitudinal or axial directions, relative to a user (such as, for example, a medical practitioner) using or holding the applicator. That is, the term “distal” is used to refer to the direction away from the medical practitioner (and toward a surface (such as, for example, a skin surface) to be treated, i.e., to which the applicator will apply a substance); and the term “proximal” is used to refer to the direction toward the user (and away from the surface to be treated). For example, the distal end of an applicator is configured to be directed toward, or even pressed against, the surface to be treated, while the proximal end extends away from the surface and toward the user. Similarly, the distal end of any portion or component of an applicator is configured to be directed or oriented toward the surface to be treated and is oriented toward the distal end of the applicator or forms or defines the distal end of the applicator. In addition, the proximal end of any portion or component of an applicator is configured to be directed or oriented away from the surface to be treated and is oriented toward the proximal end of the applicator or forms or defines the proximal end of the applicator.

It is often desirable to apply substances to surfaces. In the medical field, single-use applicators may be used to apply medical products to the skin or other tissues. Medical preparations such as, for example, skin barrier products, antiseptics, adhesion enhancing products, adhesive tape trauma protectants, and pharmaceutical agents such as analgesics may be applied as, for example, liquid solutions containing one or more substances. These solutions may be applied with saturated sponges that are attached to a blade or held with forceps. These sponges are often saturated by soaking them in open pans of solution. Sometimes, sponges with attached handles are provided in a plastic or aluminum foil laminate pouch containing enough liquid to saturate the sponges. In some products the sponges are supplied dry in a sterile “kit” with the antiseptic solutions provided in relatively thin-walled polyethylene bottles. These bottles generally have wall thickness less than about 500 microns. While inexpensive, these techniques are messy and offer little control over inadvertent dripping of the solution into areas where it is undesired.

Alternatively, devices have been developed in an attempt to prevent solution from dripping associated with these techniques, and to reduce the time required for application of the antiseptic solution. For example, liquid applicators that hold the liquid in a frangible ampoule and require additional elements to crush the ampoule and release the liquid have been developed. However, existing applicators are often complex to construct, may be difficult or cumbersome to use, and may deliver far more solution than is required for a particular treatment, particularly when a relatively small area of tissue requires substance application.

Treatment of small areas of skin with a solution is needed in some situations, such as, for example, in the application of skin barrier products to persons with ostomies, including ileostomies and colostomies. Treatment of these smaller areas may present challenges not encountered when larger treatment areas are involved. For example, because the treatment area is small, an applicator containing a small volume of solution is preferred in order to minimize wasting what may be a costly solution. Moreover, for conditions such as ostomies, such products may be applied in a nonclinical setting by the person with the condition, creating a need for a simple and easy-to-use applicator that can reliably deliver less than 5 mL (e.g., 0.2-2 mL) of a solution to a surface in need of treatment.

Applicators that can hold a small amount of substance (e.g., less than 5 mL), that allow for sterilization processes, e.g., ethylene oxide exposure, and that may be activated and ergonomically used with one hand are needed for certain applications. The present disclosure provides an applicator that, inter alia, includes a handle having an activation region that is sufficiently flexible so that the ampoule inside the applicator body may be readily fractured when the applicator is held in a single hand, while at the same time providing adequate protection to the user from injury by preventing ampoule shards from piercing the applicator body.

The present disclosure provides an applicator for applying a substance to a surface, the applicator including a frangible ampoule and a hollow body, where the hollow body includes a distal portion, a proximal portion, an exterior surface, an interior surface, and an activation region. The hollow body includes at least two longitudinal grooves on at least one of the exterior surface and the interior surface in the activation region. The hollow body further includes a first radial groove in the activation region that is on at least one of the exterior surface or the interior surface.

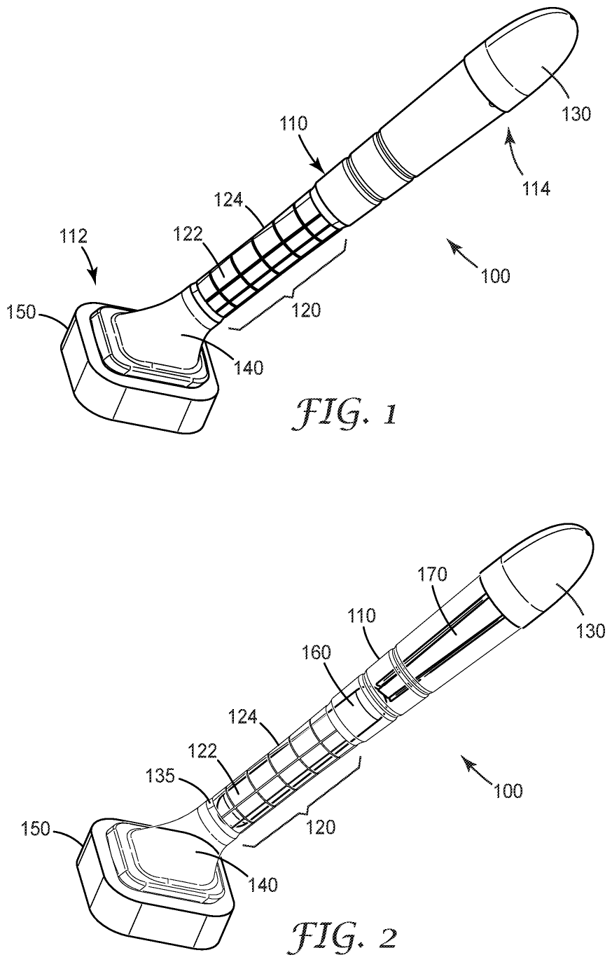

An exemplary applicator 100 according to one embodiment of the present disclosure is illustrated in FIG. 1. The applicator 100 includes a hollow body 110 and a frangible ampoule 160. Referring to FIG. 1, the hollow body 110 includes a distal portion 112, a proximal portion 114, an inner surface 116 and an outer surface 118. The hollow body 120 has a generally frustoconical geometry and may taper as shown along the longitudinal axis of the hollow body 110 from the proximal portion 114 to the distal portion 112. The taper may facilitate gripping of the applicator 100 in the hand during use. The hollow body 110 includes an activation region 120.

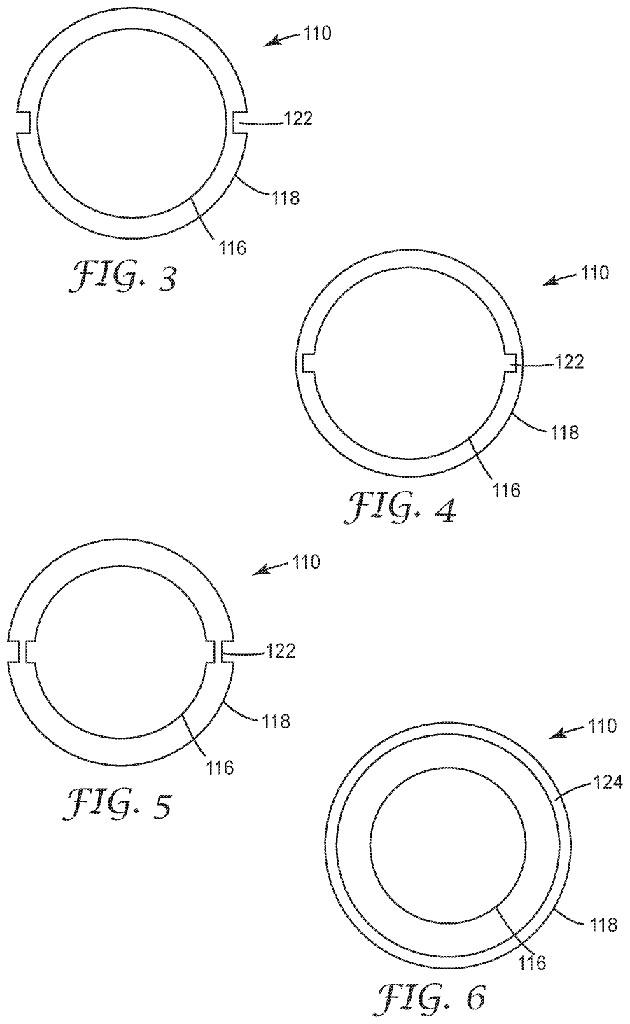

The activation region 120 includes at least two longitudinal grooves 122 on at least one of the inner surface 116 and the outer surface 118 of the activation region. FIG. 3 shows a radial cross-section through an applicator 100 activation region 120 having two longitudinal grooves 122 in the outer surface 118 where the grooves are spaced 180° apart. In some embodiments, the two longitudinal grooves 122 in the outer surface 118 may be spaced less than 180° apart, such as, for example, 140° apart, 120° apart, 90° apart, 45° apart 30° apart, or 20° apart.

In some embodiments, and as shown in FIG. 4, the activation region 120 may have two longitudinal grooves 122 in the inner surface 116 where the grooves are spaced 180° apart. In some embodiments, the two longitudinal grooves 122 in the inner surface 116 may be spaced less than 180° apart, such as, for example, 140° apart, 120° apart, 90° apart, 45° apart 30° apart, or 20° apart.

In some embodiments, and as shown in FIG. 5, the activation region 120 may have two sets of opposing longitudinal grooves 122 in the outer surface 118 and inner surface 116 where the sets of grooves 122 are 180° apart. In some embodiments, the two sets of opposing longitudinal grooves 122 in the outer surface 118 and inner surface 116 may be spaced less than 180° apart, such as, for example, 140° apart, 120° apart, 90° apart, 45° apart 30° apart, or 20° apart.

In some embodiments, the activation region 120 may include more than two longitudinal grooves 122. For example, as shown in FIGS. 1 and 2, the activation region 120 may include six longitudinal grooves 122, i.e., three grooves 122 on each side of the activation region 120.

The longitudinal grooves 122 have a width and depth sufficient to impart the desired flexibility to the activation region 120 while not reducing the thickness of the material forming the hollow body 110 to the point where it is vulnerable to puncture. In one embodiment, the hollow body 110 may be formed from a polymer (e.g., linear low-density polyethylene) having a thickness of 0.04 inch to 0.020 inch (e.g., 0.028 inch), where the activation region 120 may include a longitudinal groove 122 having a width of 0.001 inch to 0.1 inch (e.g., 0.010 inch), and a depth of 0.001 inch to 0.014 inch (e.g., 0.004 inch).

The hollow body 110 further includes a radial groove 124 in the activation region 120 that is on at least one of the inner surface 116 and the outer surface 118 of the activation region. FIG. 6 shows a radial cross-section through an applicator 100 activation region 120 having a radial groove 124 in the outer surface 118 of the applicator 100, wherein the radial groove 124 extends 180° around the body. In some embodiments, the radial groove 124 may extend less than 180° around the body, such as, for example, 160° or less, 150° or less, 140° or less, 130° or less, 120° or less, 110° or less, 100° or less, 90° or less, 80° or less, 70° or less, 60° or less, 50° or less, 40° or less, or 30° or less around the body. Commonly the radial groove 124 will intersect one or more of the longitudinal grooves 122.

The radial groove 124 has a width and depth sufficient to impart the desired flexibility to the activation region 120 while not reducing the thickness of the material forming the hollow body 110 to the point where it is vulnerable to puncture. In one embodiment, the hollow body 110 may be formed from a polymer (e.g., linear low-density polyethylene) having a thickness of 0.04 inch to 0.020 inch (e.g., 0.028 inch), where the activation region 120 may include a radial groove 124 having a width of 0.001 inch to 0.1 inch (e.g., 0.010 inch), and a depth of 0.001 inch to 0.014 inch (e.g., 0.004 inch). In some embodiments, the longitudinal grooves 122 and the radial groove 124 may have the same width and depth dimensions. In some embodiments, the longitudinal grooves 122 and the radial groove 124 may have different width and/or depth dimensions.

The hollow body 110 is configured to retain a frangible ampoule 160. Application of a force to the activation region 120 desirably results in the fracture of the frangible ampoule 160 that is contained in the hollow body 110. The force may be applied by, for example, the user holding the applicator along the palm of his/her hand and squeezing the applicator between a finger and thumb of that hand in the activation region 120.

Referring to FIG. 2, frangible ampoule 160 is positioned within hollow body 110 such that at least a portion of the frangible ampoule 160 is located in the activation region 120 so that when the activation region 128 is compressed the frangible ampoule 160 is fractured. The frangible ampoule 160 may be hermetically sealed and can contain a solution. The frangible ampoule 160 may desirably act as a protective barrier to contact between the solution contained therein and the outer environment. For example, the frangible ampoule 160 may be resistant to environmental changes encountered by the applicator 100 during manufacture, transport, and use of the applicator, such as, for example, the vacuum and chemicals, e.g., ethylene oxide, that the applicator may be exposed to during a sterilization process, conditions that could damage or destroy the solution inside the frangible ampoule 160.

Commonly, the frangible ampoule 160 may be formed of a brittle material, such as, for example, a glass. Suitable brittle materials for forming the frangible ampoule 160 include, for example, a soda-lime glass, a borosilicate glass, an onion skin borosilicate glass, a polymer, a ceramic, and combinations thereof. Such materials are desirably brittle and will fracture when compressed. This is in contrast to relatively flexible materials that would deform when compressed but which must be punctured to release the substance inside. Typically, the substance contained within the frangible ampoule 160 is released by applying external force to the applicator 100 in the activation region 120 sufficient to shatter the frangible ampoule 160. In some embodiments, score lines or other features that provide local areas of weakness in the brittle material may be included to control breaking and/or reduce the force required to break the frangible ampoule 160.

The size and shape of the frangible ampoule 160 is selected to be compatible with the dimensions of the hollow body 110 and the desired volume of substance to be applied. For example, for use in preparation for a small surgical procedure, the amount of substance in the ampoule 160 should generally be sufficient to cover an area of, such as, for example, about 10 square centimeters. For larger surgical procedures, the amount of substance in the ampoule 160 may need to be sufficient to cover at least the torso of a large person, such as, for example, at least about 500-600 square centimeters. The size of the frangible ampoule 160 may be selected to be appropriate for the volume of substance to be delivered in order to minimize product costs and waste. In addition, smaller frangible ampoules 160 may result in products with longer shelf lives. As a nonlimiting example, it has been found that solutions containing cyanoacrylate adhesives have longer shelf lives in small vials compared to larger vials even when the vials are flushed with an inert gas before sealing. Without being bound by theory, it is believed that the smaller interior surface area of a smaller frangible ampoule has fewer sites on the interior surface of the glass that can initiate polymerization of the cyanoacrylate, thereby extending the shelf life of the product.

In some embodiments, the frangible ampoule 160 is selected to apply a substance to a surface having an area such as, for example, about 5 cm² to about 200 square cm². In some embodiments, use of a smaller frangible ampoule 160 permits a frangible ampoule 160 having a thinner wall to be used, thus increasing the ease with which a frangible ampoule 160 may be fractured. In some embodiments, the frangible ampoule 160 is configured to contain, for example, from about 0.2 mL to about 2 mL (e.g., 1 mL) of a substance.

The frangible ampoule 160 contains the substance to be dispensed. Generally, a wide variety of substances can be contained within the frangible ampoule 160, with the selection of the substance influencing the selection of the materials used to construct the frangible ampoule and other parts of the applicator 100, as would be readily understood by one of ordinary skill in the art. Substances contained in the frangible ampoule may include, for example, liquids, gels, suspensions, and pastes. In some embodiments, the substance contained in the ampoule 160 is a liquid composition that includes a volatile carrier and an active agent, such as, for example, an antiseptic agent or a pharmaceutical agent. In some embodiments, the applicator 100 may be particularly useful in dispensing substances having viscosities at room temperature of less than about 10,000 cps. In other embodiments, the applicator may be particularly useful in dispensing substances having viscosities less than about 500 cps.

In some embodiments, the frangible ampoule 160 may contain an antiseptic preparation. Examples of suitable antiseptic preparations include those described in U.S. Pat. No. 4,584,192 and those described in U.S. Pat. No. 4,542,012. Other useful fluids include antiseptic preparations, such as, for example, iodophoric skin tinctures, such as DURAPREP™ Surgical Solution, commercially available from 3M Company, Saint Paul, Minn., USA. In some embodiments, the ampoule 160 may be filled with a composition that includes an antimicrobial agent such as iodine, an iodine complex (such as, for example, iodophors), chlorhexidine, chlorhexidine salts (such as, for example, chlorhexidine digluconate and chlorhexidine diacetate), or combinations thereof. Other exemplary antimicrobial agents include C2-C5 lower alkyl alcohols, fatty acid monoesters of glycerin and propylene glycol, polymers that include a (C12-C22) hydrophobe and a quaternary ammonium group, polyquaternary amines (such as, for example, polyhexamethylene biguanide), quaternary ammonium silanes, silver, silver salts (such as silver chloride), silver oxide and silver sulfadiazine, methyl, ethyl, propyl and butyl parabens, octenidine, peroxides (such as, for example, hydrogen peroxide and benzoyl peroxide), and the like, as well as combinations thereof. In other embodiments, the frangible ampoule may contain, for example, medical preparations such as skin barrier products, adhesion enhancing products, adhesive tape trauma protectants, and pharmaceutical agents such as analgesics. As a nonlimiting example, the frangible ampoule may contain a skin barrier product, such as CAVILON™ Advanced Skin Protectant, commercially available from 3M Company, Saint Paul, Minn., USA. In some embodiments, the contents of the frangible ampoule 160 include a cyanoacrylate monomer.

Referring to FIG. 2, the applicator 100 further includes filter 135, located between the frangible ampoule 160 and a head 140, the head 140 including an absorbent component 150. The filter 135 may be integrally molded to the head 140 and is provided with holes so that fluids can readily pass through it. When the frangible ampoule 160 is fractured, the filter 135 prevents shards of the ampoule 160 from reaching the absorbent component 150. Thus, the filter 135 is made sufficiently thick so as not to be pierced by the shards of the frangible ampoule 160 after the frangible ampoule 160 is broken.

The hollow body 110 may optionally include one or more ribs on the inner surface 126 of the hollow body 110. The ribs may extend longitudinally along all or part of the length of the hollow body 110 from the filter 135 to the proximal portion 114 of the body. When force is applied to activation region 120, e.g., by the hand of an operator wishing to coat a surface with the solution contained in the frangible ampoule 160, the ribs may act as stress concentrators, thereby decreasing the amount of force required to fracture the frangible ampoule 160.

The applicator further comprises cap 130. In some embodiments, and as shown in FIG. 2, the applicator optionally further comprises a spacer 170. Examples of suitable applicators 100 having a cap 130 and spacer 170 include those described in U.S. Pat. App. 62/720,682 filed Aug. 21, 2018, the contents of which are hereby incorporated by reference in their entirety. The distal end of the spacer 170 may have concave curvature. The concave curvature may be selected to complement the convex curvature of an end of an exterior surface of the frangible ampoule 160. That is, the radius of curvature of the exterior end surface of the frangible ampoule 160 and the radius of curvature of the distal end of the spacer are approximately equal. Thus, the length of spacer 170 and the curvature of its distal end may be selected to maintain the frangible ampoule 160 in a desired position, such as, for example, maintaining the frangible ampoule in the activation region 120 of the body 110.

In some embodiments, the cap 130 and the spacer 170 may be formed as a unitary piece, such as by, for example, injection molding. In other embodiments, the cap 130 and the spacer 170 may be formed as separate components which could be joined by means known in the art, such as, for example, with an adhesive, a heat seal, a spin weld, an ultrasonic weld, and combinations thereof, or the cap 130 and the spacer 170 could remain as separate components in the assembled applicator 100.

The cap 130 is adapted to be attached to the hollow body 110 by means of a snap fit. This may be accomplished by providing a retention ring (not shown) inwardly projecting from side walls adjacent to the major orifice of the cap 130 and which has a smaller inside diameter than the outside diameter of the outwardly projecting ridge of an attachment feature near the end of the proximal portion of the body. When the applicator is assembled, the retention ring of the cap is snapped over the outwardly projecting ridge of the attachment feature on the body.

To avoid creating a vacuum and restricting flow through the applicator 100, a means of maintaining atmospheric pressure in the device is preferably employed. One or more vents in the body functions to aspirate air into the internal volume of the applicator as the substance flows into the absorbent component to maintain atmospheric pressure within the device and prevent “air locking.”

Generally, the applicators 100 of the present disclosure can be made by known methods. In some embodiments, injection molding may be used. A variety of materials may be used to form the applicators. In some embodiments, applicators 100 formed of low flexural modulus materials such as low-density polyethylene may be used. For example, in some embodiments, materials having a flexural modulus of no greater than 500 Mpa, e.g., no greater than 400 MPa, or even no greater than 350 MPa may be used.

The applicator body 110, cap 130, head 140, and optional spacer 170 when present may be molded from polymers such as, for example, polyethylenes, linear low density polyethylenes, medium density polyethylenes, high density polyethylenes, branched polyethylenes, polypropylenes, and combinations thereof. In some embodiments, the applicator body 110 may be molded from a combination of linear low-density polyethylene and medium density polyethylene.

The absorbent component 150 may be formed of any suitable porous substance such as, for example, a sponge, a woven material, a nonwoven material, a screen, a mesh, and combinations thereof. Materials suitable for use as the absorbent component 150 may include, for example, polyester polyurethane and polyester polyether open-cell foams. The absorbent component 150 may be attached to the head 140 at the distal end 112 of the hollow body 110 and may, among other functions, aid in control of the flow rate and distribution of the contents of the frangible ampoule 160 after the contents have been released from the frangible ampoule 160.

In some embodiments, the absorbent component 110 may be attached to the head 140 at the distal end 112 of the hollow body 110 by, for example, hot plate welding. In other embodiments, the absorbent component 110 may be attached to hollow body 120 by other means, such as, for example, an adhesive, a spin weld, an ultrasonic weld, or combinations thereof.

A substance, such as those described above, may be applied to a surface with an applicator as described in the present disclosure by releasing the substance from the frangible ampoule, e.g., the user holding the applicator along the palm of his/her hand and squeezing the applicator between a finger and thumb of that hand in the activation region such that the substance flows into the absorbent component when the frangible ampoule is fractured, and then applying the substance to a surface by pressing the absorbent component to the surface.

In some embodiments, the surface comprises mammalian tissue such as, for example, skin, bone, cartilage, enamel, dentin, cementum, dental pulp, gum tissue, mucous membrane, organ tissue, epithelial tissue, ocular tissue, tympanic tissue, connective tissue, muscular tissue, nervous tissue, and combinations thereof. In some embodiments, the mammalian tissue may include a perforation such as, for example, incision, an abrasion, a laceration, a puncture, an orifice, and combinations thereof.

Various modifications and alterations of this invention will become apparent to those skilled in the art without departing from the scope and spirit of this disclosure. All cited references, patents, and patent applications in the above application for letters patent are herein incorporated by reference in their entirety in a consistent manner. In the event of inconsistencies or contradictions between portions of the incorporated references and this application, the information in the preceding description shall control. The preceding description, given in order to enable one of ordinary skill in the art to practice the claimed disclosure, is not to be construed as limiting the scope of the disclosure, which is defined by the claims and all equivalents thereto.

1. An applicator for applying a substance to a surface, the applicator comprising:
a hollow body, wherein the hollow body includes a distal portion, a proximal portion, an outer surface, an inner surface, and an activation region between the distal portion and proximal portion,
wherein the hollow body includes two longitudinal grooves on at least one of the outer surface and the inner surface in the activation region; and
wherein the hollow body has a first radial groove in the activation region that is on at least one of the outer surface and the inner surface.

2. The applicator of claim 1, wherein the longitudinal grooves are at least 90° apart.

3. The applicator of claim 1, wherein the longitudinal grooves are 180° apart.

4. The applicator of claim 1, wherein the hollow body includes at least six longitudinal grooves on the outer surface in the activation region.

5. The applicator of claim 1, wherein the radial groove extends at least 90° around the body.

6. The applicator of claim 1, wherein the radial groove extends 180° around the body.

7. The applicator of claim 1, wherein the hollow body includes at least five radial grooves on the outer surface in the activation region.

8. The applicator of claim 1, wherein the applicator further comprises a cap, a spacer, a head, a frangible ampoule, and an absorbent component.

9. The applicator of claim 1, wherein the applicator comprises a polymer selected from the group consisting of a polyethylene, a linear low-density polyethylene, a medium-density polyethylene, a high-density polyethylene, a branched polyethylene, a polypropylene, and combinations thereof.

10. The applicator of claim 1, wherein the applicator body comprises a combination of linear low-density polyethylene and medium-density polyethylene.
